(19) United States
(12) Patent Application Publication
(10) Pub. No.: US 2024/0188619 A1
(43) Pub. Date: Jun. 13, 2024

(54) HASHISH PRODUCT AND INDUSTRIAL PROCESS FOR MAKING SAME

(71) Applicant: HEXO OPERATIONS INC., GATINEAU (CA)

(72) Inventor: Jamie Savard, Gatineau (CA)

(21) Appl. No.: 18/286,690

(22) PCT Filed: Apr. 14, 2022
(86) PCT No.: PCT/CA2022/050588

§ 371 Date: Oct. 12, 2023

Related U.S. Application Data
(60) Provisional application No. 63/175,940, filed on Apr. 16, 2021.

(51) Int. Cl. A24B 15/16; A24B 15/18; A61K 31/00

(52) U.S. Cl. CPC A24B 15/16; A24B 15/18; A61K 31/658

(57) ABSTRACT

The present disclosure relates to hashish products and processes for manufacturing same. The processes comprise pressing partly decarboxylated isolated cannabis trichomes to obtain a cohesive mass of isolated cannabis trichomes. The hashish product comprises a not negligible content in one or more acid form of one or more cannabinoid(s) and can therefore be characterized as having a more extended shelf life. Known prior art processes for manufacturing hashish products have a batch-like approach that involves completely decarboxylating cannabinoid(s) by applying heat to isolated trichomes via the pressing plates or to the pressed trichomes after the pressing step followed by a second pressing step. This batch-like approach to manufacturing hash can be labor intensive, reduce volume throughput and negatively affect overall efficiency of the hashish production process, which increases costs and complicates production.

### CROSS REFERENCE TO RELATED APPLICATION

The present application claims the benefit of U.S. provisional patent application Ser. No. 63/175,940 filed on Apr. 16, 2021. The contents of the above-referenced document are incorporated herein by reference in their entirety.

### TECHNICAL FIELD

This application generally relates to the field of hashish products as well as industrial methods of manufacturing same.

### BACKGROUND

With stage-wise legalization of cannabis-based consumer products in Canada and eventually in various other areas in the world, advancements in extraction technology, industrial scale production and accessibility to a wide variety of forms have accelerated to fulfill emerging demands. Hashish (or hash) is one example of a cannabis-based product, typically used for recreational or medicinal (i.e., health and wellness) purposes, for which there is an increasing consumer demand.

Hashish is a cohesive mass of isolated cannabis trichomes. Cannabis trichomes can be isolated in several ways. For example, trichomes can be isolated from cannabis plants by hand, by mechanical beating of the plants or by submersing the cannabis plants in icy water and then using sieving to isolate the trichomes. Alternatively, mechanical separation may be used to isolate cannabis trichomes from cannabis plants, such as with motorized tumblers as described for example in WO 2019/161509.

Traditionally, hashish is obtained by pressing isolated cannabis trichomes manually. In an industrial setting, however, manual pressing is hardly scalable and affords poor yield—instead, hashish is obtained by pressing isolated cannabis trichomes in a mechanical press.

Using a mechanical press to press the isolated trichomes produces a cohesive mass from the isolated trichomes (i.e., hashish product). When the pressing occurs in a mould, the pressed hashish product takes the form afforded by the mould. For example, the isolated trichomes can be pressed in a mould affording the shape of individual “bricks”. During pressing, heat may be applied via the pressing plates to cause a release of resin from the trichomes and decarboxylate the cannabinoids (activate the acid form of the cannabinoids). Alternatively, heat may be applied to the pressed trichomes after the pressing step for substantially the same purposes. Typically, hashish manufacturers perform a second pressing step after such heating to further ensure cohesiveness of the hashish product. From a production perspective, this batch-like approach to manufacturing hash (e.g., applying heat on a per hashish unit basis after or during pressing) can be labor intensive, reduce volume throughput and negatively affect overall efficiency of the hashish production process, which increases costs and complicates production.

Considering the above, it would be highly desirable to be provided with a hashish product having desired properties while being provided with methods of making same that would at least partially alleviate the disadvantages of the pressing methods discussed above.

### SUMMARY

This Summary is provided to introduce a selection of concepts in a simplified form that are further described below in the Detailed Description. This Summary is not intended to identify key aspects or essential aspects of the claimed subject matter.

Broadly stated, in some embodiments, the present disclosure relates to a process for making a hashish product, comprising a) providing pre-treated isolated cannabis trichomes, the pre-treated isolated cannabis trichomes comprising a cannabis oil layer on at least a portion of a surface thereof; and b) pressing the pre-treated isolated cannabis trichomes under conditions to form the hashish product, the hashish product being a cohesive mass of the pre-treated isolated cannabis trichomes, and the hashish product comprising a not negligible content in acid form of one or more cannabinoid(s).

Broadly stated, in some embodiments, the present disclosure relates to a process for making a hashish product, comprising a) providing partially decarboxylated isolated cannabis trichomes; and b) pressing the pre-treated isolated cannabis trichomes under conditions to form the hashish product, the hashish product being a cohesive mass of the partially decarboxylated isolated cannabis trichomes. Advantageously, the hashish product comprising a not negligible content in acid form of one or more cannabinoid(s).

In some embodiments, the process includes one or more of the following features
the step a) comprises pre-heating isolated cannabis trichomes under conditions to obtain partial decarboxylation of the one or more cannabinoid(s).
the step a) comprises (i) pre-heating cannabis material under conditions to obtain partial decarboxylation of the one or more cannabinoid(s) and (ii) isolating cannabis trichomes therefrom.
the pre-heating is performed at a temperature of from about 70° C. to about 130° C., preferably from about 80° C. to about 120° C., more preferably about 120° C.
the pre-heating is performed for a duration of from about 10 minutes to about 60 minutes, preferably from about 20 minutes to about 35 minutes.
the pre-heating is performed to obtain a decarboxylation level of the one or more cannabinoid(s) of from about 30% to about 90%, preferably from about 40% to about 80%, more preferably from about 50% to about 70%, even more preferably about 60%.
the pre-heating is performed to obtain a ratio of a decarboxylated to acid form content of the one or more cannabinoid(s), by weight, of from about 100:1 to about 1:100, preferably from about 1:4 to about 50:1, more preferably from about 1:3 to about 5:1.
the not negligible content in the acid form of the one or more cannabinoid(s) is of no less than 1 wt. %, preferably of no less than 3 wt. %, even more preferably of no less than 5 wt. %.
the not negligible content in the acid form of the one or more cannabinoid(s) is of up to about 30 wt. %, preferably up to about 25 wt. %, more preferably up to about 20 wt. %.
the acid form of the one or more cannabinoid(s) comprises tetrahydrocannabinolic acid (THC-A), cannabidiolic acid (CBD-A), or THC-A and CBD-A.
further comprising incorporating water to the pre-treated isolated cannabis trichomes prior to step b) to have a water content of about 20 wt. % or less, preferably between about 5 wt. % and about 15 wt. %, more preferably from about 10 wt. % to about 15 wt. %.
the water is incorporated in the form of liquid, ice, steam, or a combination thereof.
the pressing is performed with a single pressing step.
the pressing is performed with a pressure of about 100 psi or more.
the pressure is from about 100 psi to about 3200 psi, preferably from about 300 psi to about 1500 psi, more preferably from about 500 psi to about 1250 psi.
the pressing is performed for a time duration of about 1 minute or more, preferably from about 1 minute to about 10 minutes, more preferably from about 3 minutes to about 7 minutes, even more preferably from about 4 minutes to about 6 minutes.
the pre-treated isolated cannabis trichomes are from a single cannabis strain.
the pre-treated isolated cannabis trichomes are from a plurality of cannabis strains.
the pre-treated isolated cannabis trichomes are pre-treated kief.
the partially decarboxylated isolated cannabis trichomes are from a single cannabis strain.
the partially decarboxylated isolated cannabis trichomes are from a plurality of cannabis strains.
the partially decarboxylated isolated cannabis trichomes are partially decarboxylated kief.

Broadly stated, in some embodiments, the present disclosure relates to a hashish product comprising a cohesive mass of isolated cannabis trichomes and a not negligible content in acid form of one or more cannabinoid(s) and made by the process described herein.

Broadly stated, in some embodiments, the present disclosure relates to a hashish product comprising a cohesive mass of isolated cannabis trichomes, a not negligible content in acid form of one or more cannabinoid(s) and having one or more of the following properties as determined in a three-point bending test: a higher limit of stiffness of about 3200 g/mm, a higher limit of hardness of about 2500 g, and a higher limit of toughness of about 8000 g*mm.

In some embodiments, the hashish product includes one or more of the following features:
the acid form of the one or more cannabinoid(s) comprises tetrahydrocannabinolic acid (THC-A), cannabidiolic acid (CBD-A), or both THC-A and CBD-A.
the not negligible content in the acid form of the one or more cannabinoid(s) is of no less than 1 wt. %, preferably of no less than 3 wt. %, even more preferably of no less than 5 wt. %.
the not negligible content in the acid form of the one or more cannabinoid(s) is of up to about 30 wt. %, preferably up to about 25 wt. %, more preferably up to about 20 wt. %.
the isolated cannabis trichomes are from a single cannabis strain.
the isolated cannabis trichomes are from a plurality of cannabis strains.
the isolated cannabis trichomes are kief.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments.

### BRIEF DESCRIPTION OF THE DRAWINGS

A detailed description of specific exemplary embodiments is provided herein below with reference to the accompanying drawings in which:

FIG. 1A illustrates a non-limiting flowchart example of a process for making a hashish product in accordance with an embodiment of the present disclosure;

FIGS. 1B-C illustrate non-limiting flowchart examples of steps for obtaining partly decarboxylated isolated trichomes in accordance with embodiments of the present disclosure;

FIG. 2A illustrates a non-limiting schematic of a setting for performing a three-point bend test;

FIG. 2B illustrates a non-limiting example of a diagram output from the three-point bend test of FIG. 2A;

FIG. 3A illustrates a non-limiting schematic of a setting for performing a puncture test;

FIG. 3B illustrates a non-limiting example of a diagram output from the puncture test of FIG. 3A;

FIGS. 4A-E illustrate physical properties of hashish products produced in accordance with example 4 of the present disclosure;

FIGS. 5A-E illustrate physical properties of hashish products produced in accordance with example 7 of the present disclosure;

FIGS. 6A-E illustrate physical properties of hashish products produced in accordance with example 10 of the present disclosure;

FIGS. 7A-E illustrate physical properties of hashish products produced in accordance with example 12 of the present disclosure.

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

A detailed description of one or more embodiments is provided below along with accompanying figures that illustrate principles of the disclosure. The invention is described in connection with such embodiments, but the invention is not limited to any embodiment. The scope of the invention is limited only by the claims. Numerous specific details are set forth in the following description to provide a thorough understanding of the invention. These details are provided for the purpose of non-limiting examples and the invention may be practiced according to the claims without some or all these specific details. Technical material that is known in the technical fields related to the invention has not been described in detail so that the disclosure is not unnecessarily obscured.

The present inventors have surprisingly and unexpectedly discovered that at least some of the problems and shortcomings discussed above with respect to existing pressing methods for hashish production can be resolved and/or alleviated using the herein described process.

For example, the present inventors have surprisingly and unexpectedly discovered that pressing pre-treated isolated cannabis trichomes to obtain a cohesive mass alleviates the negative impact of manufacturing hashish products in the batch-like approach discussed previously, while achieving the desired hashish physical attributes, e.g., in terms of malleability, pliability, and/or crumbliness. Indeed, as discussed previously, the existing batch-like approach to manufacturing hash currently requires applying heat during the pressing step via the pressing plates or to the cohesive mass after the pressing step. Further, manufacturers often perform a second pressing step on the cohesive mass after such heat/press step to ensure good cohesiveness of the hashish product. This batch-like multiple steps approach to manufacturing hashish can be labor intensive, reduce volume throughput and negatively affect overall efficiency of the hashish production process, which increases costs and complicates production.

The herein described approach provides the technical advantage of avoiding this batch-like multiple steps approach by pretreating the raw materials instead of the finished product.

For example, large amounts of isolated trichomes or cannabis material can be pre-treated in a single step instead of treating the finished hashish product on a per unit basis, thus avoiding the bottleneck of treating the finished product on a per unit basis.

For example, pre-treatment of isolated trichomes or cannabis material avoids the risk of overcooking the more expensive hashish product that may occur in the batch-like heat treatment of the prior art, as overcooked isolated trichomes or cannabis material may still be used in other applications whereas overcooked hashish products may not.

For example, pre-treatment of isolated trichomes or cannabis material allows better inventory management as the pre-treated isolated trichomes or cannabis material can be stored for later use and may be used in more than one product type.

Further, the herein described hashish product includes not negligible amounts of the acid form of one or more cannabinoid(s). This in turn, allows the hashish product to have an extended shelf life in comparison to hashish products of the prior art that have fully decarboxylated cannabinoids. Indeed, it is known that the acid form of cannabinoids is more stable over time than decarboxylated form thereof and, as such, cannabinoid potency of the hashish product of the present disclosure can be maintained over extended periods of time comparatively.

These and other advantages may become apparent to the person of skill in view of the present disclosure.

### Hashish Product

The hashish product of the present disclosure comprises a cohesive mass of isolated cannabis trichomes.

As used herein, the term “cannabis trichomes” generally refers to crystal-shaped outgrowths or appendages (also called resin glands) on cannabis plants typically covering the leaves and buds. Trichomes produce hundreds of known cannabinoids, terpenes, and flavonoids that make cannabis strains potent, unique, and effective.

As used herein, the term “isolated cannabis trichomes” refers to trichomes that have been separated from cannabis plant material using any method known in the art. The details of various methods for separating trichomes from the cannabis plant are well-known in the art. For example, and without wishing to be limiting in any manner, the isolated cannabis trichomes may be obtained by a chemical separation method or may be separated by manual processes like dry sifting or by water extraction methods. Solvent-less extraction methods can include mechanical separation of trichomes from the plant, such as sieving through a screen by hand or in motorized tumblers (see for example WO 2019/161509), or by submerging the cannabis plants in icy water (see for example US2020/0261824, which is herein incorporated by reference) and agitating to separate the trichomes from the plant and drying the trichomes. Because of inherent limitations to existing separation methods, some plant matter or other foreign matter can be present in isolated cannabis trichomes.

Isolated cannabis trichomes obtained by mechanical separation of trichomes from the cannabis plant biomass is typically referred to as “kief” (also “keef” or “kif”) and has a powdery appearance. In preferred embodiments of the present disclosure, the isolated cannabis trichomes is in the form of kief.

The isolated cannabis trichomes forming the hashish product of the present disclosure may originate from one or more than one strain of cannabis plant. It is known amongst consumers of hashish and other cannabis products that using isolated cannabis trichomes produced from more than one strain of cannabis plant allows a user to tune the psychoactive entourage effect obtained by consuming the product. The mixing of cannabis plant strains may also allow to adjust the final concentration of a component of the product, for example but not limited to the cannabinoid content. Additionally, use of more than one strain allows for improved product and waste management—important in commercial production.

As used herein, the term “cannabis” generally refers to a genus of flowering plants that includes a number of species. The number of species is currently being disputed. There are three different species that have been recognized, namely Cannabis sativa, Cannabis indica and Cannabis ruderalis. Hemp, or industrial hemp, is a strain of the Cannabis sativa plant species that is grown specifically for the industrial uses of its derived products. In terms of cannabinoids content, hemp has lower concentrations of tetrahydrocannabinol (THC) and higher concentrations of cannabidiol (CBD).

The hashish product of the present disclosure comprises one or more cannabinoid(s). As used herein, the term “cannabinoid” generally refers to any chemical compound that acts upon a cannabinoid receptor such as CB1 and CB2. Examples of cannabinoids include, but are not limited to, cannabichromanon (CBCN), cannabichromene (CBC), cannabichromevarin (CBCV), cannabicitran (CBT), cannabicyclol (CBL), cannabicyclovarin (CBLV), cannabidiol (CBD, defined below), cannabidiolic acid (CBD-A), cannabidiol monomethylether (CBDM), cannabidiol-C4 (CBD-C4), cannabidiorcol (CBD-C1), cannabidiphorol (CBDP), cannabidivarin (CBDV), cannabielsoin (CBE), cannabifuran (CBF), cannabigerol (CBG), cannabigerol monomethylether (CBGM), cannabigerolic acid (CBGA), cannabigerovarin (CBGV), cannabinodiol (CBND), cannabinodivarin (CBVD), cannabinol (CBN), cannabinol methylether (CBNM), cannabinol propyl variant (CBNV), cannabinol-C2 (CBN-C2), cannabinol-C4 (CBN-C4), cannabiorcol (CBN-C1), cannabiripsol (CBR), cannabitriol (CBO), cannabitriolvarin (CBTV), cannabivarin (CBV), dehydrocannabifuran (DCBF), Δ7-cis-iso tetrahydrocannabivarin, tetrahydrocannabinol (THC, defined below), Δ9-tetrahydrocannabinolic acid (THC-A) including either or both isomers 2-COOH-THC (THCA-A) and 4-COOH-THC (THCA-B), Δ9-tetrahydrocannabiorcol (THC-C1), tetrahydrocannabivarinic acid (THCVA), tetrahydrocannabivarin (THCV), ethoxy-cannabitriolvarin (CBTVE), trihydroxy-Δ9-tetrahydrocannabinol (triOH-THC), 10-ethoxy-9hydroxy-46a-tetrahydrocannabinol, 8,9-dihydroxy-Δ6a-tetrahydrocannabinol, 10-oxo-46a-tetrahydrocannabionol (OTHC), 3,4,5,6-tetrahydro-7-hydroxy-α-α-2-trimethyl-9-n-propyl-2, 6-methano-2H-1-benzoxocin-5-methanol (OH-iso-HHCV), Δ6a, 10a-tetrahydrocannabinol (Δ6a, 10a-THC), Δ8-tetrahydrocannabivarin (Δ8-THCV), Δ9-tetrahydrocannabiphorol (Δ9-THCP), Δ9-tetrahydrocannabutol (Δ9-THCB), derivatives of any thereof, and combinations thereof. Further examples of suitable cannabinoids are discussed in at least WO2017/190249 and U.S. Patent Application Pub. No. US2014/0271940, which are each incorporated by reference herein in their entirety.

Cannabidiol (CBD) means one or more of the following compounds: Δ2-cannabidiol, Δ5-cannabidiol (2-(6-isopropenyl-3-methyl-5-cyclohexen-I-yl)-5-pentyl-1,3-benzenediol); Δ4-cannabidiol (2-(6-isopropenyl-3-methyl-4-cyclohexen-I-yl)-5-pentyl-1,3-benzenediol); Δ3 cannabidiol (2-(6-isopropenyl-3-methyl-3-cyclohexen-I-yl)-5-pentyl-1,3-benzenediol); Δ3,7-cannabidiol (2-(6-isopropenyl-3-methylenecyclohex-I-yl)-5-pentyl-1,3-benzenediol); Δ2-cannabidiol (2-(6-isopropenyl-3-methyl-2-cyclohexen-I-yl)-5-pentyl-1,3-benzenediol); Δ1-cannabidiol (2-(6-isopropenyl-3-methyl-I-cyclohexen-I-yl)-5-pentyl-1,3-benzenediol); and Δ6-cannabidiol (2-(6-isopropenyl-3-methyl-6-cyclohexen-I-yl)-5-pentyl-1,3-benzenediol). In a preferred embodiment, and unless otherwise stated, CBD means Δ2-cannabidiol.

Tetrahydrocannabinol (THC) means one or more of the following compounds: Δ8-tetrahydrocannabinol (Δ8-THC), Δ8-tetrahydrocannabivarin (Δ8-THCV), Δ9-cis-tetrahydrocannabinol (cis-THC), Δ9-tetrahydrocannabinol (Δ9-THC), Δ10-tetrahydrocannabinol (Δ10-THC), Δ9-tetrahydrocannabinol-C4 (THC-C4), Δ9-tetrahydrocannabinolic acid-C4 (THCA-C4), synhexyl (n-hexyl-Δ3THC). In a preferred embodiment, and unless otherwise stated, THC means one or more of the following compounds: Δ9-tetrahydrocannabinol and Δ8-tetrahydrocannabinol.

A cannabinoid may be in an acid form or a non-acid form, the latter also being referred to as the decarboxylated form since the non-acid form can be generated by decarboxylating the acid form.

Advantageously, the hashish product of the present disclosure comprises a content of an acid form of one or more cannabinoid(s) which is not negligible.

As used herein, the term “not negligible” in combination with the concept of the acid form content of the one or more cannabinoids refers to a content which is sufficient to provide the herein described desired physical properties of the hashish product. For example, an acid form content of the one or more cannabinoids which is not negligible can be of no less than about 1 wt. %, preferably of no less than about 3 wt. %, more preferably of no less than about 5 wt. %. For example, the acid form content of the one or more cannabinoids which is not negligible can be of up to about 35 wt. %, up to about 30 wt. %, up to about 25 wt. %, or up to about 20 wt. %, including any values therein or in a range of values defined by the aforementioned values.

The endogenous content of one or more cannabinoid(s) in cannabis strains has been reported in the literature. For example, Coogan in Analysis of the cannabinoid content of strains available in the New Jersey Medicinal Marijuana Program. J Cannabis Res 1, 11 (2019) has reported that various strains of cannabis flower from licensed operators in the New Jersey Medicinal Marijuana Program can be combined in three broad groups of strains: those with <1 wt. % CBD-A and with THC-A concentration from 10 to 30 wt. %; those with both THC-A and CBD-A concentrations in the 5-10 wt. % range; and those with <1 wt. % THC-A and with CBD-A concentration >10 wt. %. It is thus within the reach of the person skilled in the art to use the teachings of the present disclosure to assess the level of decarboxylation required for a given cannabis material or sub-part thereof to obtain the herein desired hashish product properties.

In one embodiment, the hashish product of the present disclosure contains the one or more cannabinoid(s) in an amount sufficient for the user to experience a desired effect when consuming the product. For example, the hashish product may comprise the one or more cannabinoid(s) in an amount of from about 5 wt. % to about 90 wt. %, or any value therebetween, or in a range of values defined by any values therebetween. For example, the hashish product may comprise the one or more cannabinoid(s) in an amount of up to about 90 wt. %, up to about 80 wt. %, up to about 70 wt. %, up to about 60 wt. %, or up to about 50 wt. %, or up to about 40 wt. %, or up to about 30 wt. % or any value therebetween, or in a range of values defined by any values therebetween. For example, the hashish product of the present disclosure may include THC, CBD, CBG, CBN, or any combinations thereof, in similar or different amounts.

The hashish product of the present disclosure may include one or more terpenes.

As used herein, the term “terpenes” generally refers to refer to a class of chemical components comprised of the fundamental building block of isoprene, which can be linked to form linear structures or rings. Terpenes may include hemiterpenes (single isoprenoid unit), monoterpenes (two units), sesquiterpenes (three units), diterpenes (four units), sesterterpenes (five units), triterpenes (six units), and so on. At least some terpenes are expected to interact with, and potentiate the activity of, cannabinoids. Examples of terpenes known to be extractable from cannabis include aromadendrene, bergamottin, bergamotol, bisabolene, borneol, 4-3-carene, caryophyllene, cineole/eucalyptol, p-cymene, dihydroj asmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, 4-terpineol, terpinolene, and derivatives thereof. Additional examples of terpenes include nerolidol, phytol, geraniol, alpha-bisabolol, thymol, genipin, astragaloside, asiaticoside, camphene, beta-amyrin, thujone, citronellol, 1,8-cineole, cycloartenol, and derivatives thereof. Further examples of terpenes are discussed in US Patent Application Pub. No. US2016/0250270, which is incorporated herein by reference in its entirety for all purposes.

The hashish product can be characterized in several ways, such as in terms of cannabinoid content, terpenes content, water content or physical properties. For example, the hashish product can be characterized in terms of stiffness, hardness, toughness, or a combination thereof, which reflect its malleability, pliability, and/or crumbliness.

As used herein the term “stiffness” refers to the amount of resistance with which a hashish sample opposes a change in the shape under application of a force and is therefore representative of the pliability of the hashish product.

As used herein the term “hardness” refers to the maximum force required for a hashish sample to reach the breaking point and is therefore representative of how easily the hashish product may be cut or separated.

As used herein the term “toughness” refers to the ability of a hashish sample to absorb energy and plastically deform without breaking. Toughness is a measure of the likelihood that the hashish product deforms rather than fractures under an applied force.

In some embodiments, the textural consistency, pliability and/or crumbliness of the hashish product can be characterized with material properties of the product, for example as determined with a three-point bending test using a Texture Analyzer. In such embodiments, the hashish product may be characterized as having one or more of the following: a higher limit of stiffness of about 3200 g/mm as measured in the three-point bend test, a higher limit of hardness of about 2500 g as measured in the three-point bend test, and a higher limit of toughness of about 8000 g*mm as measured in the three-point bend test.

For example, the stiffness can be of from about 200 g/mm to about 3200 g/mm, including any ranges there in-between or any values therein. For example, the stiffness can be of about 200 g/mm, about 250 g/mm, about 300 g/mm, about 350 g/mm, about 400 g/mm, about 450 g/mm, 500 g/mm, about 550 g/mm, about 600 g/mm, about 650 g/mm, about 700 g/mm, about 750 g/mm, about 800 g/mm, about 850 g/mm, about 900 g/mm, about 950 g/mm, about 1000 g/mm, about 1050 g/mm, about 1100 g/mm, about 1150 g/mm, about 1200 g/mm, about 1250 g/mm, about 1300 g/mm, about 1350 g/mm, about 1400 g/mm, about 1450 g/mm, about 1500 g/mm, about 1550 g/mm, about 1600 g/mm, about 1650 g/mm, about 1700 g/mm, about 1750 g/mm, about 1800 g/mm, about 1850 g/mm, about 1900 g/mm, about 1950 g/mm, about 2000 g/mm, about 2100 g/mm, about 2200 g/mm, about 2300 g/mm, about 2400 g/mm, about 2500 g/mm, about 2600 g/mm, about 2700 g/mm, about 2800 g/mm, about 2900 g/mm, about 3000 g/mm, about 3100 g/mm, or about 3200 g/mm including any value there in-between or any ranges with any of these values as range limits.

For example, the hardness can be of from about 250 g to about 2500 g, including any ranges there in-between or any values therein. For example, the hardness can be of about 250 g, about 500 g, about 750 g, about 1000 g, about 1150 g, about 1200 g, about 1250 g, about 1300 g, about 1350 g, about 1400 g, about 1450 g, about 1500 g, about 1550 g, about 1600 g, about 1650 g, about 1700 g, about 1750 g, about 1800 g, about 1850 g, about 1900 g, about 1950 g, about 2000 g, about 2100 g, about 2200 g, about 2300 g, about 2400 g, or about 2500 g, including any value there in-between or any ranges with any of these values as range limits.

For example, the toughness can be of from about 1500 g*mm to about 8000 g*mm, including any ranges there in-between or any values therein. For example, the toughness can be of about 1500 g*mm, about 2500 g*mm, about 3500 g*mm, about 4500 g*mm, about 5500 g*mm, about 6500 g*mm, about 7500 g*mm, or about 8500 g*mm, including any value there in-between or any ranges with any of these values as range limits.

In some embodiments, the moisture content in the hashish product of the present disclosure, achieved through addition of water to the isolated trichomes, can be of about 5 wt. % or more. For example, the moisture content can be of from 10 wt. % to about 50 wt. %, or any value therebetween, or in a range of values defined by any values therebetween, as described in PCT Application PCT/CA2020/051733, which is hereby incorporated by reference in its entirety.

### Use of Hashish Products

Hashish products are typically used for recreational and/or medicinal uses. For example, hashish products can be used to achieve a desired effect in a user, such as a psychoactive effect, a physiological effect, or a treatment of a condition. By “psychoactive effect”, it is meant a substantial effect on mood, perception, consciousness, cognition, or behavior of a subject resulting from changes in the normal functioning of the nervous system. By “physiological effect”, it is meant an effect associated with a feeling of physical and/or emotional satisfaction. By “treatment of a condition”, it is meant the treatment or alleviation of a disease or condition by absorption of cannabinoid(s) at sufficient amounts to mediate the therapeutic effects.

The terms “treating”, “treatment” and the like are used herein to mean obtaining a desired pharmacologic and/or physiologic effect. The effect may be prophylactic, in terms of completely or partially preventing a disease, condition, or symptoms thereof, and/or may be therapeutic in terms of a partial or complete cure for a disease or condition and/or adverse effect, such as a symptom, attributable to the disease or disorder. “Treatment” as used herein covers any treatment of a disease or condition of a mammal, such as a dog, cat or human, preferably a human.

In certain embodiments, the disease or condition is selected from the group consisting of pain, anxiety, an inflammatory disorder, a neurological disorder, a psychiatric disorder, a malignancy, an immune disorder, a metabolic disorder, a nutritional deficiency, an infectious disease, a gastrointestinal disorder, and a cardiovascular disorder. Preferably the disease or condition is pain. In other embodiments, the disease or condition is associated with the feeling of physical and/or emotional satisfaction.

In the context of recreational use, the “effective amount” administered and rate and time-course of administration, will depend on the desired effect associated with a feeling of physical and/or emotional satisfaction in the subject.

In the context of health and wellness use, the “effective amount” administered and rate and time-course of administration will depend on the nature and severity of the disease or condition being treated and typically also takes into consideration the condition of the individual subject, the method of administration and the like.

### Manufacturing Process

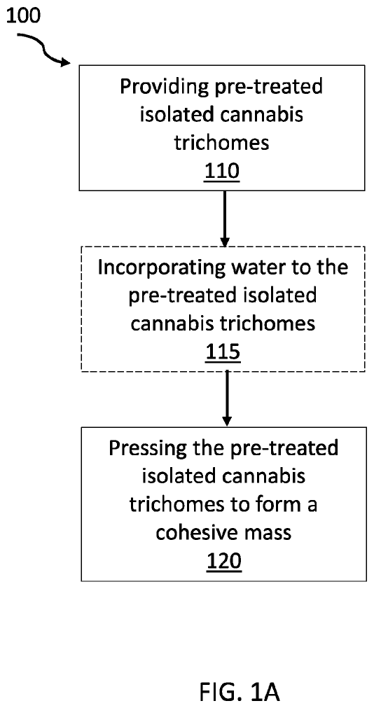

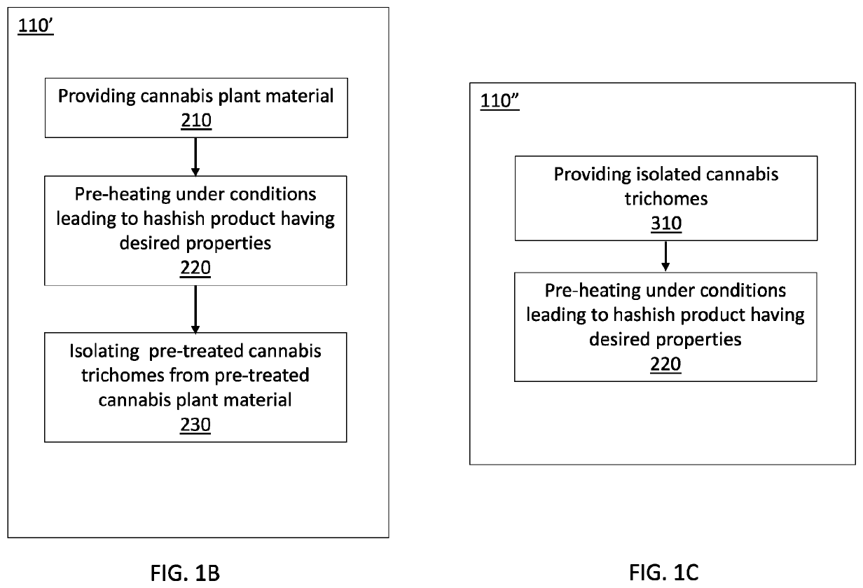

FIG. 1A is a non-limiting flowchart of a process 100 for making a hashish product in accordance with an embodiment of the present disclosure. The process 100 comprises a first step 110 of providing pre-treated isolated cannabis trichomes.

In one non-limiting example, the pre-treated isolated cannabis trichomes may include trichomes isolated from a single cannabis strain. In another non-limiting example, the pre-treated isolated cannabis trichomes may include trichomes isolated from a plurality of distinct cannabis strains, which may have different respective cannabinoid(s) and/or terpene(s) content. The choice of one over the other may be driven by practical considerations, such as but not limited to inventory management considerations, the desired cannabinoid content of the hashish product, the desired user experience, and the like.

The pre-treated isolated cannabis trichomes may be obtained in several ways.

The producer implementing the process 100 may obtain the pre-treated isolated cannabis trichomes from another producer. The step 110 may thus include a sub-step of obtaining the pre-treated isolated cannabis trichomes from another producer (not shown in figures).

Alternatively, the producer implementing the process 100 may obtain the pre-treated isolated cannabis trichomes via at least one of the following variants of step 110.

FIG. 1B is a first variant step 110′ which includes starting from pre-treated cannabis plant material to isolate the pre-treated cannabis trichomes therefrom. In this variant, a first step 210 includes providing cannabis plant material comprising cannabis trichomes. The cannabis plant material may comprise cannabis flowers/buds, cannabis trim, cannabis leaves, or any combination thereof. The producer implementing the first variant step 110′ may also produce the cannabis plant material or may obtain the cannabis plant material from another producer. In a second step, the cannabis plant material is pre-treated with a pre-heating step 220 under conditions leading to ultimately obtaining a hashish product with the desired properties, such as in terms of malleability, crumbliness, and/or pliability. In a subsequent step 230, cannabis trichomes are isolated from the pre-treated cannabis plant material thus resulting in the pre-treated isolated cannabis trichomes. As discussed previously, various processes for isolating cannabis trichomes from cannabis plant material are known and as such, will not be further described here.

FIG. 1C is a second variant step 110″ which includes starting from isolated cannabis trichomes to obtain the pre-treated isolated cannabis trichomes. In this variant, a first step 310 includes providing isolated cannabis trichomes. The producer implementing the second variant step 110″ may also produce the isolated cannabis trichomes or may obtain the isolated cannabis trichomes from another producer. In a second step, the isolated cannabis trichomes are pre-treated with the pre-heating step 220 described above.

The present inventors have surprisingly discovered that the conditions for performing the pre-heating step 220 can be optimized using the decarboxylation level as a biomarker to monitor the extend of pre-heating that is suitable for a given situation. Indeed, the present inventors have discovered that when the pre-heating step 220 is performed under conditions leading to partial decarboxylation, one can ultimately obtain a hashish product having the desired properties, such as in terms of malleability, crumbliness, and/or pliability. In other words, the pre-heating step 220 is preferably performed under conditions such that the content in acid form of one or more cannabinoid(s) of the pretreated material (i.e., cannabis plant material or isolated cannabis trichomes) is not negligeable.

In some embodiments, the variant steps 110′ and/or 110″ may be performed at a first location while the remaining steps of process 100 may be performed at a second location, where the first and second locations may be within the same licensed producer site or within different licensed producer sites. In some embodiments, all steps of process 100 may be performed at the same location.

The pre-treatment of the isolated cannabis trichomes can be monitored in several ways.

In some embodiments, the pre-treatment of the isolated cannabis trichomes may be performed to obtain a desired decarboxylation level. For example, the decarboxylation level can be from about 30% to about 90%, or any value therebetween, or in a range of values defined by any values therebetween. For example, the level of decarboxylation can be from about 40% to about 80%, from about 50% to about 70%, or about 60%. The decarboxylation level can be determined, for example, by comparing the initial content in the acid form of a specific cannabinoid to the remaining content in the acid form of the specific cannabinoid after the pre-heating step 220. For example, if the initial content in the acid form of a specific cannabinoid is 30 wt. % and the remaining content in the acid form of that specific cannabinoid obtained after the pre-heating step 220 is 15 wt. %, it means that the decarboxylation level is 50% (i.e., half of the initial content in the acid form of a specific cannabinoid was decarboxylated).

In some embodiments, the pre-treatment of the isolated cannabis trichomes may be performed to obtain a desired ratio of decarboxylated vs. acid form (wt. %:wt. %) of a specific cannabinoid. For example, the ratio may be of from about 100:1 to about 1:100, or any value therebetween, or in a range of values defined by any values therebetween. For example, from about 1:4 to about 50:1, or from about 1:3 to about 5:1.

The content in the acid form and the decarboxylated form of a specific cannabinoid can be determined using suitable methods known to the person skilled in the art, such as but not limited to Gas Chromatography/Mass Spectrometry (GC/MS), High Performance Liquid Chromatography (HPLC), Gas Chromatography/Flame Ionization Detection (GC/FID), Fourier transform infrared (FT-IR) spectroscopy, and the like. Various suitable methods are described, for example, in Formato et al., “(−)-Cannabidiolic Acid, a Still Overlooked Bioactive Compound: An Introductory Review and Preliminary Research.” Molecules. 2020 Jun. 5; 25(11):2638.

The process 100 may further comprises an optional step 115 of incorporating water to the pre-treated isolated cannabis trichomes prior to the pressing step, as further described below. Water may be incorporated in the form of steam, liquid, ice, or a combination. The water incorporated may be distilled, reverse osmosis and/or microfiltered water. In some embodiments, water may be incorporated to have a total water content of about 20 wt. % or less. For example, a total water content of from about 5 wt. % to about 15 wt. % or any value therebetween, or in a range of values defined by any values therebetween. For example, a total water content of about 15 wt. % or less, about 14 wt. % or less, about 13 wt. % or less, about 12 wt. % or less, about 11 wt. % or less, about 10 wt. % or less. For example, a total water content of from about 10 wt. % to about 15 wt. %, from about 10 wt. % to about 12 wt. %.

It will be readily appreciated that the total water content of the isolated cannabis trichomes may be adjusted to any desired/target value, for example to obtain the desired physical properties of the hashish product (e.g., in terms of malleability, pliability, and/or crumbliness), as further described below. In other words, the relative amount of water being incorporated into the pre-treated isolated cannabis trichomes at optional step 115 may be dependent upon several factors, as further described below, such as the pressing conditions (i.e., pressure and/or duration, as further described below), the conditions for performing the pre-heating step 220 and/or the desired physical properties of the hashish product.

In some embodiments, the conditions for performing the pre-heating step 220 may include time duration, temperature, or a combination thereof.

For example, the pre-heating temperature may be from about 70° C. to about 130° C., or any value therebetween, or in a range of values defined by any values therebetween. For example, from about 80° ° C. to about 120° C., or about 120° C.

For example, the pre-heating time may be from about 10 minutes to about 60 minutes, or any value therebetween, or in a range of values defined by any values therebetween. For example, from about 20 minutes to about 40 minutes, or about 25 minutes, or about 30 minutes, or about 35 minutes.

It will be readily apparent to the person skilled in the art that various combinations and permutations of pre-heating temperature and pre-heating time may be used to achieve identical, similar or substantially similar decarboxylation content of a specific cannabinoid (e.g., a longer pre-heating time with a lower pre-heating temperature, a higher pre-heating temperature with a shorter pre-heating time, etc.) and it is well within the skills of that person to select and implement such combinations and permutations that will achieve the desired result, in view of the herein described teachings. For example, it will be also readily apparent to the person skilled in the art that the pre-heating time and the pre-heating temperature may be selected based on the strain of cannabis plant, the type of cannabis plant material (where applicable), the method of isolation of the cannabis trichomes (where applicable), and the like.

In some non-limiting examples, the pre-heating temperature can be of about 120° C. and the pre-heating time may be of from about 10 to about 40 minutes; in some cases, the pre-heating temperature can be of about 120° C. and the pre-heating time may be of from about 20 to about 35 minutes; in some cases, the pre-heating temperature can be of about 100° C. and the pre-heating time may be of from about 60 to about 80 minutes; in some cases, the pre-heating temperature can be of about 80° C. and the pre-heating time may be of from about 120 minutes or more; or any other range or value within such ranges.

In some embodiments, the pre-heating step 220 may be performed in any suitable heating apparatus (e.g., an oven), which may be equipped with proper ventilation if desired.

In some embodiments, the herein described acid form of the one or more cannabinoid(s) comprises THC-A, CBD-A, or a combination. Preferably, the specific cannabinoid assessed to monitor the level of decarboxylation described herein is THC-A.

### Pressing Pre-Treated Trichomes

Returning to FIG. 1, the process 100 further comprises a step 120 of pressing the pre-treated isolated cannabis trichomes. Such pressing may be performed mechanically with an industrial press, for example. The pre-treated isolated cannabis trichomes are pressed at step 120 under conditions to form a cohesive mass of the pre-treated isolated cannabis trichomes.

The conditions to form the cohesive mass of the pre-treated isolated cannabis trichomes at the pressing step 120 comprise at least a pressure (i.e., the pressure that is being exerted onto the pre-treated isolated cannabis trichomes to form the cohesive mass) and a duration (i.e., the time during which the pressure is being exerted).

For example, the pressure being applied at the pressing step 120 may be between about 100 psi or more. For example, a pressure of about 3200 psi or less, in some cases between about 300 psi and about 1500 psi, in some cases between 500 psi and about 1250 psi, or any other range or value within such ranges. In some embodiments, the pressure in the pressing step 120 may be expressed by a certain load (in US tons, for example) exerted on a surface area corresponding to a mould dimension. For example, for a 2″ by 4″ pressing plate (or mould) having a pressing area of 8 square inches, a load of 3.9 US tons corresponds to a pressure of about 975 psi, a load of 5 US tons corresponds to a pressure of about 1250 psi, a load of 10 tons corresponds to a pressure of about 2500 psi. Other mould dimensions and loads are possible and may be adopted by the person skilled the art without departing from the scope of the present disclosure.

For example, the pressure being applied at the pressing step 120 may be applied for a duration of about 1 minute or more. For example, between about 1 minute and about 10 minutes, between about 3 minutes and about 7 minutes, between about 4 minutes and about 6 minutes, or about 5 minutes, or any range or value within such ranges.

It will be readily apparent to the person skilled in the art that various combinations and permutations of pressure and duration can be used to form the cohesive mass (e.g., a longer or shorter duration or pressure, a longer duration with a lower pressure, a higher pressure with a shorter duration, etc.) and it is well within the skills of that person to identify such combinations and permutations. For example, the pressure can be of about 1200 psi and the duration can be of from about 4 to about 6 minutes; the pressure can be of about 1200 psi and the duration can be of about 5 minutes; the pressure can be of about 600 psi and the duration can be of from about 4 to about 6 minutes; the pressure can be of about 600 psi and the duration can be of about 5 minutes, or any range or value within such ranges.

It will be also readily apparent to the person skilled in the art that the conditions of the pressing step 120 may vary according to specifics of the pre-treated isolated cannabis trichomes being pressed at step 120, such as but not limited to the strain of cannabis from which the pre-treated cannabis trichomes were isolated, the cannabinoid and/or terpene content of the pre-treated isolated cannabis trichomes, the overall volume of the pre-treated isolated cannabis trichomes being pressed, the water content of the pre-treated isolated cannabis trichomes, and the like. Again, it is well within the skills of that person to identify suitable combinations and permutations of pressure and duration.

In other embodiments, the pressing step 120 is a first pressing step and the process 100 further comprises a second pressing step (not shown) after the first pressing step 120, although a single pressing step is preferred. Much like the first pressing step 120, the second pressing step can also be performed under conditions to form a cohesive mass of the pre-treated isolated cannabis trichomes which comprise a second pressure and a second duration. The second pressing step can optionally further include a pressing temperature.

For example, the second pressure may be of from about 325 psi to about 1,600 psi; in some cases, the second pressure may be of from about 550 psi to about 1,350 psi; in some cases, the second pressure may be of about 800 psi to about 1,300 psi, or any other range or value within such ranges.

For example, the second duration may be of about 1 minute or less; in some cases, the second duration may be of about 30 seconds or less; in some cases, the second duration may be of about 20 seconds or less, and in some cases even less.

For example, the optional pressing temperature of the second pressing step may be of from about 50° C. to about 80° C.; in some cases, the pressing temperature of the second pressing step may be of about 70° C., or any other range or value within such range.

In some embodiments, the pre-treated isolated cannabis trichomes are pressed at step 120 in a mould having a prescribed shape and/or dimensions to form the cohesive mass. The cohesive mass may thus exhibit a shape and/or dimensions that generally conforms to that of the mould. Advantageously, the mould may have a desirable shape and/or dimensions, such as but not limited to square-like, ovoid-like, tablet-like, ball-like, and the like.

Advantageously, the pre-treated isolated cannabis trichomes do not need to undergo the pressing step 120 on the same day that the cannabis trichomes are pre-heated at step 220. In some examples, the pre-treated isolated cannabis trichomes could be stored for a period of up to 48 hours, or up to 24 hours, or up to 12 hours before being subjected to the pressing step 120 without significantly deteriorating the physical attributes (for example in terms of malleability, pliability, and/or crumbliness) of the hashish product. This facilitates the operation and logistics of the hashish production process 100 as there is less risk of producing degraded hashish products in cases where the pre-treated isolated cannabis trichomes cannot be pressed at step 120 on the same day as the pre-heating step 220.

Without being bound by any theory, it is believed that the herein described pre-treatment causes cannabis oils and/or resin to ooze out from the cannabis trichomes at least partially, which then form an oil layer on at least a surface thereof. This oil layer can be qualitatively observed upon performing the herein described pre-treatment step as the resulting pre-treated isolated trichomes have a “moist” appearance thereafter. It is believed that this oil layer may facilitate the adhesion of the isolated trichomes one to another during the subsequent pressing step without requiring the need for additional heating and/or pressing steps, as is typically performed in batch-like pressing processes of the prior art. It is believed that causing the proper balance of oil amounts oozing out from the cannabis trichomes is key to ultimately obtaining the desired hashish properties. The present inventors have herein described monitoring the decarboxylation level as one manner of correlating the extent of pre-treatment to the desired oil amounts oozing out from the cannabis trichomes.

### Three-Point Bend Test

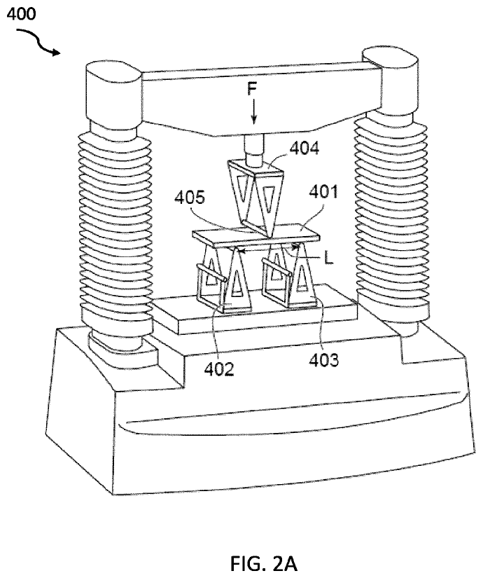

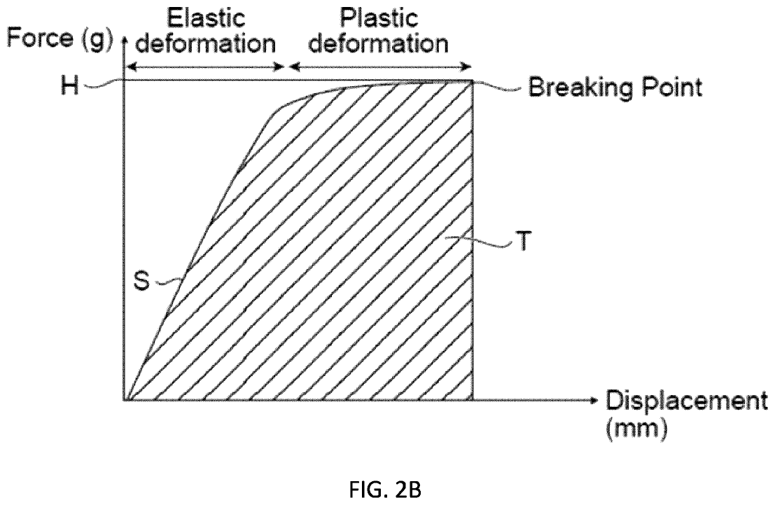

FIG. 2A is a non-limiting example of a three-point bend test employed to determine physical properties of the hashish product.

During the test, the force applied to and the displacement of the probe are recorded. A force-over displacement graph is typically generated and usually begins with an ascending linear section that corresponds to elastic (reversible) deformation, then most samples show a curved section that shows plastic (irreversible) deformation. Different samples will give different load-distance responses—stronger and stiffer samples show higher forces, brittle samples break before any plastic deformation occurs and tough samples show a large area under the curve corresponding to a large amount of energy required for deformation.

The test procedure using a Texture Analyzing device 400 is as follows:
a. a 20 mm by 20 mm sample of hashish product 401 having a height of about 5-6 mm was placed on two support anvils 402/403 of the Texture Analyzing device 400, which anvils were distanced by a predetermined length (L),
b. a gradually descending probe 404 attached to a 100 kg load cell was landed on the center point 405 of the sample 401 while exerting a controlled vertical force F on the sample 401 until the sample 401 started to bend (elastic deformation) followed by a plastic deformation and eventually broke apart. A force-over-displacement graph was generated by the Texture analyzer software.

A non-limiting example of a force-over-displacement graph obtained from the three-point bend test is shown in FIG. 2B, where the applied force F (expressed in grams, “g”) is plotted against the probe displacement (expressed in mm). The resulting graph includes a linear ascending elastic deformation zone and a plastic deformation zone. The slope of the curve in the linear elastic deformation zone is equivalent to Stiffness (as shown by “S” in FIG. 2B). The maximum force beyond which the sample breaks (breaking point) is equivalent to Hardness (as shown by “H” in FIG. 2B). The area under the curve (expressed as g*mm) is equivalent to Toughness (as shown by “T” in FIG. 2B).

The three-point bend test can be performed with a Texture analyzer, such as the TA.XT Plus or TA.XT2 available from Stable MicroSystems (Surrey, United Kingdom), the TA-XT2i/5 texture analyzer from Texture Technologies Corp. (Scarsdale, N.Y), or any other texture analyzing instrument known to a person of skill in the art.

### Puncture Test

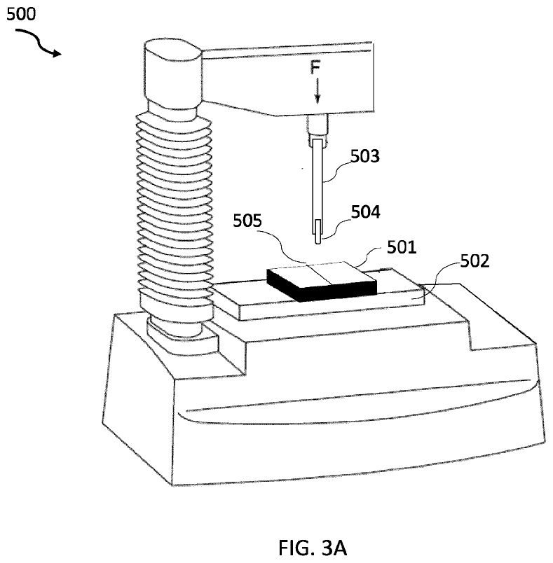

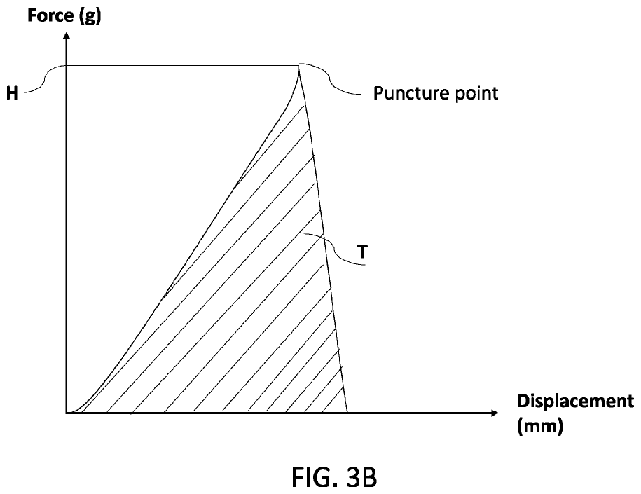

FIG. 3A. is a non-limiting example of a puncture test employed to determine physical properties of the hashish product.

During the test, the force applied to and the displacement of the probe are recorded. A force-over displacement graph is typically generated and usually begins with an ascending linear section that corresponds to elastic (reversible) deformation which reaches to a maximum peak as sign of sample puncture, then samples show a sharp descending section that shows plastic (irreversible) deformation post-puncture. Different samples will give different load-distance responses—stronger and harder samples show higher forces, softer samples puncture faster. Tough samples show a large area under the curve corresponding to a large amount of energy required for deformation (puncture).

The test procedure using a Texture Analyzing device 500 is as follows:
a. a 20 mm by 20 mm sample of hashish product 501 having a height of about 5-6 mm was placed on a flat surface 502 of the Texture Analyzing device 500,
b. a gradually descending probe 503 (comprising a 2 mm tip 504) attached to a 100 kg load cell was landed on the center point 505 of the sample 501 while exerting a controlled vertical force F on the sample 501 until the sample 501 is punctured. A force-over-displacement graph was generated by the Texture analyzer software.

A non-limiting example of a force-over-displacement graph obtained from the puncture test is shown in FIG. 3B, where the applied force F (expressed in grams, “g”) is plotted against the probe displacement (expressed in mm). The resulting graph includes an ascending linear elastic deformation zone reaching a peak followed by a sharp descending zone. The maximum force beyond which the sample punctures (puncture point) is equivalent to Hardness (as shown by “H” in FIG. 3B). The area under the curve (expressed as g*mm) is equivalent to Toughness (as shown by “T” in FIG. 3B).

The puncture test can be performed with a Texture analyzer, such as the TA.XT Plus or TA.XT2 available from Stable MicroSystems (Surrey, United Kingdom), or any other texture analyzing instrument known to a person of skill in the art.

### EXAMPLES

The following examples are for illustrative purposes only and are not meant to limit the scope of the compositions and methods described herein.

### Example 1: Pre-Heating at 120° C.

In this example, a batch (Kief ID BBI-088) of isolated cannabis trichomes (NLxBB cannabis strain) was placed in an oven at 120° C. for pre-heating same. Samples were retrieved from the oven pre-determined pre-heating time duration (namely at 20 min, 40 min, 60 min, and 80 min). The decarboxylation level of the samples was measured with high performance liquid chromatography (HPLC) using THC-A and THC content. The results are summarized in Table 1.

TABLE 1

| | Pre- | | | | |
|---|---|---|---|---|---|
| | heating | | | Total | |
| Sample | duration | [THC] | [THC-A] | [THC] | Ratio |
| # | (min) | (wt. %) | (wt. %) | (wt. %) | THC:THC-A |
| 1 | 0 | 0.30 | 33.7 | 32.60 | 1:100 |
| 2 | 20 | 21.34 | 13.56 | 33.23 | 1.5:1 |
| 3 | 40 | 32.87 | 0.69 | 33.48 | 50:1 |
| 4 | 60 | 33.81 | 0.28 | 34.05 | 100:1 |
| 5 | 80 | 33.46 | 0.17 | 33.61 | 150:1 |

Table 1 shows that pre-heating isolated NLxBB cannabis trichomes at 120° C. for 20 min affords partly decarboxylated isolated cannabis trichomes. In contrast, pre-heating at 120° C. for 40 min was sufficient to obtain substantially complete decarboxylation.

### Example 2: Pressing Pre-Treated Isolated Cannabis Trichomes from Example 1

In this example, hashish products were made by pressing the pre-treated isolated cannabis trichomes samples from Example 1. The pressing parameters were the following: mould having a size of 2.5×2.5 inches, 2-press step including a first press with a press load of 7800 lbs for 5 minutes at 20° C. and a second press with a press load of 7800 lbs for 20 seconds at 70° C., water added to kief to have a total of 10 wt. % water. The resulting hashish products were characterized in terms of textural properties. The results are summarized in Table 2.

TABLE 2

| Sample # | Hashish textural properties |
|---|---|
| 2 | Good malleability |
| 3 | Too soft |
| 4 | Too soft |
| 5 | Too soft |

It was observed that pressing pre-treated isolated cannabis trichomes from the sample pre-heated at 120° ° C. for 20 min (sample #2) afforded a hashish product with good malleability. In contrast, pressing pre-treated isolated cannabis trichomes from the sample pre-heated at 120° C. for 40 min (sample #3) as well from samples pre-heated for longer (samples #4 and #5) produced hashish products with soft texture.

### Example 3: Pre-Heating at 120° C.

In this example, a batch (Kief ID RND0004150-01) of isolated cannabis trichomes (SL cannabis strain) was placed in an oven at 120° C. for pre-heating same. Samples were retrieved from the oven pre-determined pre-heating time duration (namely at 5 min, 10 min, 15 min, 20 min, 25 min, and 30 min). The decarboxylation level of the samples was measured with HPLC using THC-A and THC content. The results are summarized in Table 3.

TABLE 3

| | Pre- | | | | |
|---|---|---|---|---|---|
| | heating | | | Total | |
| Sample | duration | [THC] | [THC-A] | [THC] | Ratio |
| # | (min) | (wt. %) | (wt. %) | (wt. %) | THC:THC-A |
| 1 | 0 | 5.5 | 27.52 | 29.64 | 1:5 |
| 2 | 5 | 5.79 | 28.69 | 30.95 | 1:5 |
| 3 | 10 | 5.84 | 26.85 | 29.38 | 1:5 |
| 4 | 15 | 6.47 | 27.34 | 30.45 | 1:4 |
| 5 | 20 | 7.59 | 26.9 | 31.18 | 1:3 |
| 6 | 25 | 22.77 | 7.68 | 29.5 | 3:1 |
| 7 | 30 | 25.7 | 5.1 | 30.17 | 5:1 |

Table 3 shows that pre-heating isolated SL cannabis trichomes for at 120° C. from 15 min (sample #4) up to 30 min (sample #7) results in partial decarboxylation.

### Example 4: Pressing Pre-Treated Isolated Cannabis Trichomes from Example 3

In this example, hashish products were made by pressing the pre-treated isolated cannabis trichomes samples from Example 3. The pressing parameters were identical to those of Example 2. The resulting hashish products were characterized in terms of visual and textural properties. The results are summarized in Table 4.

TABLE 4

| Sample # | Hashish product description |
|---|---|
| 1 | Dark tan, crumbly |
| 2 | Dark tan, crumbly and hard |
| 3 | Dark brown, crumbly |
| 4 | Dark brown, crumbly |
| 5 | Dark brown, almost crumbly but mostly self-adhesive |
| 6 | Dark brown, self-adhesive without crumbling |
| 7 | Dark brown, self-adhesive without crumbling |

From Table 4, it was observed that pressing pre-treated isolated SL cannabis trichomes from the sample pre-heated at 120° C. from a time duration of from 20 min (sample #5) up to 30 min (sample #7) afforded hashish products having desired physical properties, namely self adhesive properties and with minimal or no crumbling.

### Example 5: Physical Characterization of Hashish Products from Example 4

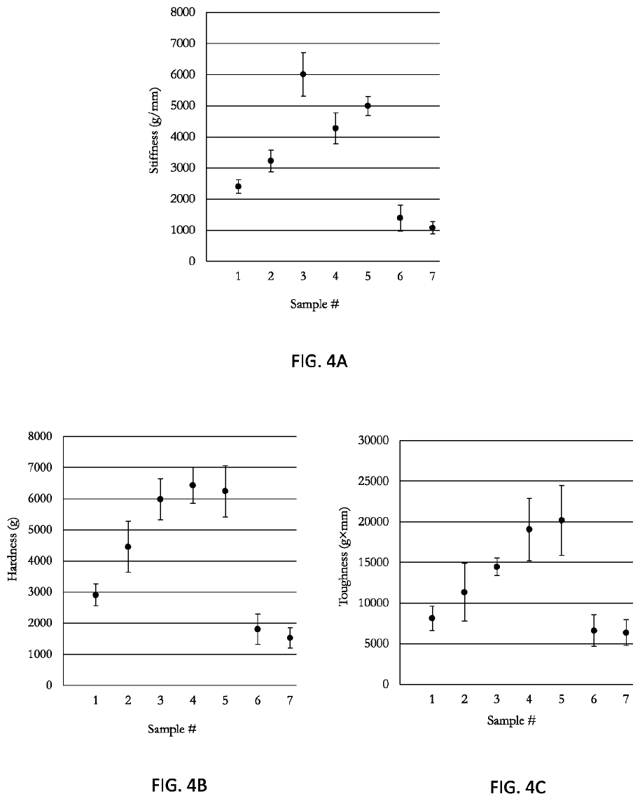

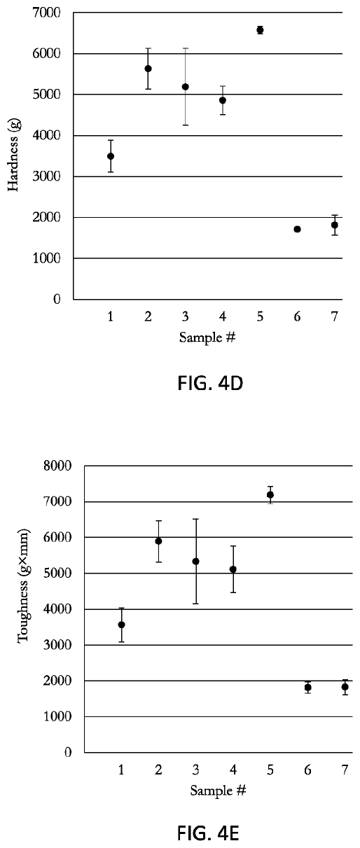

In this Example, physical properties of the hashish products manufactured in Example 4 were determined with the three-point bend test and puncture test. Results for the three-point bend test are shown in Table 5A and FIGS. 4A-C.

TABLE 5A

| | Three-Point Bend Test | | | |
|---|---|---|---|---|
| | Stiffness | Hardness | Toughness | Sample Height |
| Sample # | (g/mm) | (g) | (g*mm) | (mm) |
| 1 | 2406 ± 218 | 2909 ± 356 | 8136 ± 1474 | 5.94 ± 0.16 |
| 2 | 3231 ± 352 | 4459 ± 818 | 11330 ± 3554 | 6.17 ± 0.15 |
| 3 | 6011 ± 698 | 5986 ± 663 | 14475 ± 1103 | 6.05 ± 0.08 |
| 4 | 4279 ± 497 | 6434 ± 576 | 19077 ± 3860 | 6.36 ± 0.37 |
| 5 | 4994 ± 309 | 6244 ± 826 | 20205 ± 4324 | 5.97 ± 0.04 |
| 6 | 1396 ± 418 | 1810 ± 481 | 6607 ± 1936 | 5.58 ± 0.31 |
| 7 | 1078 ± 198 | 1529 ± 323 | 6366 ± 1579 | 5.64 ± 0.21 |

Results for the puncture test are shown in Table 5B and FIGS. 4 D-E.

TABLE 5B

| | Puncture Test | | |
|---|---|---|---|
| | Hardness | Toughness | Sample Height |
| Sample # | (g) | (g*mm) | (mm) |
| 1 | 3494 ± 387 | 3568 ± 471 | 5.73 ± 0.07 |
| 2 | 5630 ± 497 | 5898 ± 579 | 6.05 ± 0.14 |
| 3 | 5189 ± 942 | 5334 ± 1180 | 5.76 ± 0.2 |
| 4 | 4860 ± 344 | 5113 ± 657 | 5.68 ± 0.61 |
| 5 | 6572 ± 89 | 7188 ± 238 | 5.75 ± 0.11 |
| 6 | 1710 ± 58 | 1818 ± 157 | 5.54 ± 0.48 |
| 7 | 1815 ± 240 | 1830 ± 210 | 5.94 ± 0.05 |

Tables 5A-B and FIG. 4A-E show that pressing pre-treated isolated cannabis trichomes pre-heated at 120° C. for a time duration of from 20 min (sample #5) up to 30 min (sample #7) afforded a hashish product with desired physical properties, namely stiffness, hardness and toughness as measured in the three-point bend test. Indeed, these hashish products were characterized as having one or more of the following: a higher limit of stiffness of about 3200 g/mm, a higher limit of hardness of about 2500 g, and a higher limit of toughness of about 8000 g*mm, as measured in the three-point bend test.

### Example 6: Pre-Heating at 100° C.

In this example, a batch (Kief ID BBI-087) of isolated cannabis trichomes (NLxBB cannabis strain) was placed in an oven at 100° C. for pre-heating same. Samples were retrieved from the oven pre-determined pre-heating time duration (namely 20 min, 30 min, 40 min, 50 min, 60 min, 70 min and 80 min). The decarboxylation level of the samples was measured with HPLC using THC-A and THC content. The results are summarized in Table 6.

TABLE 6

| | Pre- | | | | |
|---|---|---|---|---|---|
| | heating | [THC] | [THC-A] | Total [THC] | Ratio |
| Sample # | duration | (wt. %) | (wt. %) | (wt. %) | THC:THC-A |
| 1 | 20 | 5.12 | 27.37 | 29.13 | 1:5 |
| 2 | 30 | 7.87 | 24.91 | 29.71 | 1:3 |
| 3 | 40 | 8.78 | 22.85 | 28.82 | 1:2 |
| 4 | 50 | 10.12 | 20.21 | 27.84 | 1:2 |
| 5 | 60 | 14.11 | 16.20 | 28.32 | 1:1 |
| 6 | 70 | 15.60 | 13.87 | 27.76 | 1:1 |
| 7 | 80 | 19.57 | 9.10 | 27.55 | 2:1 |

Table 6 shows that pre-heating isolated NLxBB cannabis trichomes at 100° C. from 20 min (sample #1) up to 80 min (sample #7) results in partial decarboxylation.

### Example 7: Pressing Pre-Treated Isolated Cannabis Trichomes from Example 6

In this example, hashish products were made by pressing the pre-treated isolated cannabis trichomes samples from Example 6. The pressing parameters were the following: mould size of 2.5×2.5 inch, one step pressing with press load of 7800 lbs for 5 minutes at 25° C., water added to kief to have a total of 10 wt. % water. The resulting hashish products were characterized in terms of visual and textural properties. The results are summarized in Table 7.

TABLE 7

| Sample | |
|---|---|
| # | Hashish product description |
| 1 | Green/brown in color (not black), easily breaks |
| 2 | Brown, uniform with slight shine, hard and easily breaks |
| 3 | Dark brown, partially bends but still hard |
| 4 | Dark brown, improved bending compared to 40 min, but still |
| | hard |
| 5 | Dark brown, improved bending compared to 50 min, but still |
| | hard |
| 6 | Brown/black in color, Acceptable malleability, and slightly |
| | tough, acceptable shine |
| 7 | Brown/black in color, Acceptable malleability, and |
| | acceptable shine |

Table 7 show that pressing pre-treated isolated cannabis trichomes pre-heated at 100° ° C. for a time duration of 50 min (sample #5) afforded a hashish product with improved bending compared to that one obtained with sample #4, whereas acceptable malleability was obtained with pressing pre-treated isolated cannabis trichomes pre-heated at 100° C. for a time duration of 80 min (sample #8).

### Example 8: Physical Characterization of Hashish Products from Example 7

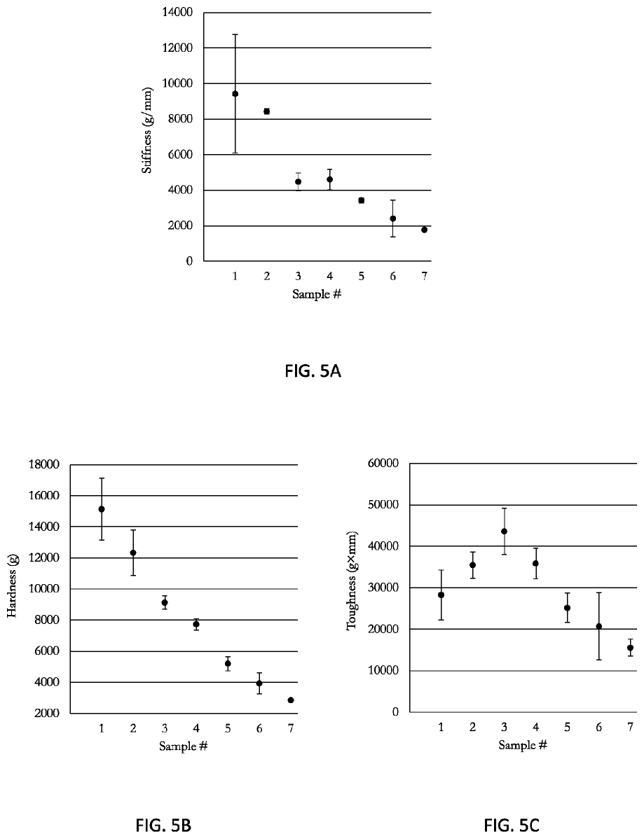

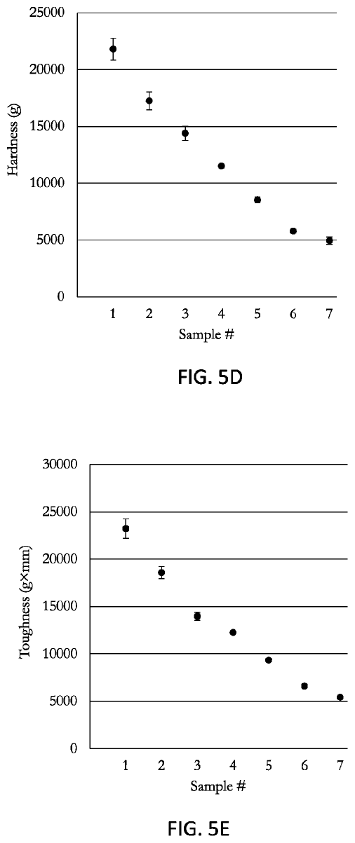

In this Example, physical properties of the hashish products manufactured in Example 7 were determined with the three-point bend test and puncture test. Results for the three-point bend test are shown in Table 8A and FIGS. 5A-C.

TABLE 8A

| | Three Point Bend Test | | | |
|---|---|---|---|---|
| | | | | Sam- |
| Sam- | | | | ple |
| ple | Stiffness | Hardness | Toughness | Height |
| # | (g/mm) | (g) | (g*mm) | (mm) |
| 1 | 9418 ± 3331 | 15136 ± 1999 | 28269 ± 6021 | 5.26 ± 0.1 |
| 2 | 8434 ± 141 | 12339 ± 1459 | 35483 ± 3156 | 5.14 ± 0.12 |
| 3 | 4470 ± 501 | 9129 ± 429 | 43587 ± 5588 | 5.32 ± 0.2 |
| 4 | 4602 ± 585 | 7729 ± 366 | 35849 ± 3690 | 4.81 ± 0.14 |
| 5 | 3426 ± 135 | 5198 ± 465 | 25173 ± 3523 | 4.67 ± 0.15 |
| 6 | 2405 ± 1038 | 3928 ± 675 | 20683 ± 8102 | 4.49 ± 0.27 |
| 7 | 1767 ± 36 | 2856 ± 3 | 15567 ± 2022 | 5.211 ± 0.001 |

Results for the puncture test are shown in Table 8B and FIG. 5 D-E.

TABLE 8B

| | Puncture Test | | |
|---|---|---|---|
| | Hardness | Toughness | Sample Height |
| Sample # | (g) | (g × mm) | (mm) |
| 1 | 21804 ± 972 | 23226 ± 1037 | 5.5 ± 0.02 |
| 2 | 17243 ± 796 | 18580 ± 629 | 5.59 ± 0.01 |
| 3 | 14390 ± 637 | 13968 ± 461 | 5.73 ± 0.18 |
| 4 | 11510 ± 186 | 12262 ± 17 | 5.42 ± 0.06 |
| 5 | 8511 ± 260 | 9341 ± 171 | 5.43 ± 0.02 |
| 6 | 5781 ± 148 | 6599 ± 31 | 5.18 ± 0.05 |
| 7 | 4935 ± 319 | 5419 ± 8 | 5.43 ± 0.13 |

Tables 8A-B show that pressing isolated cannabis trichomes pre-heated at 100° C. for 80 min affords a hashish product with the desired physical properties, in terms of one or more of the following: a higher limit of stiffness of about 3200 g/mm as measured in the three-point bend test, a higher limit of hardness of about 2500 g as measured in the three-point bend test, and a higher limit of toughness of about 8000 g*mm as measured in the three-point bend test.

### Example 9: Pre-Heating at 80° C.

In this example a batch (Kief ID BBI-087) of isolated cannabis trichomes (NLxBB cannabis strain) was placed in an oven at 80° C. for pre-heating same. Samples were retrieved from the oven pre-determined pre-heating time duration (namely 20 min, 30 min, 40 min, 50 min, 60 min, 70 min, 80 min, 90 min and 120 min). The decarboxylation level of the samples was measured with HPLC using THC-A and THC content. The results are summarized in Table 9.

TABLE 9

| | Heating | | | | |
|---|---|---|---|---|---|
| Sample | duration | [THC] | [THC-A] | Total [THC] | Ratio |
| # | (min) | (wt. %) | (wt. %) | (wt. %) | THC:THC-A |
| 1 | 20 | 3.12 | 29.37 | 28.88 | 1:9 |
| 2 | 30 | 3.28 | 31.21 | 30.65 | 1:10 |
| 3 | 40 | 3.39 | 28.21 | 28.14 | 1:9 |
| 4 | 50 | 4.00 | 29.61 | 29.97 | 1:7 |
| 5 | 60 | 4.14 | 28.89 | 29.48 | 1:7 |
| 6 | 70 | 4.11 | 28.08 | 28.73 | 1:7 |
| 7 | 80 | 4.23 | 27.73 | 28.56 | 1:6 |
| 8 | 90 | 4.69 | 26.92 | 28.30 | 1:6 |
| 9 | 120 | 5.12 | 26.38 | 28.26 | 1:5 |

Table 9 shows that pre-heating isolated NLxBB cannabis trichomes for 50 min at 80° C. (sample #4) initiates partial decarboxylation. Partial decarboxylation is still obtained with pre-heating time up to 120 min (sample #9).

### Example 10: Pressing Pre-Treated Isolated Cannabis Trichomes from Example 9

In this example, hashish products were made by pressing the pre-treated isolated cannabis trichomes samples from Example 9. The pressing parameters were identical to those of Example 2. The resulting hashish products were characterized in terms of visual properties.

TABLE 10

| Sample # | Hashish product description |
|---|---|
| 1 | Hard, easily breaks |
| 2 | Hard, easily breaks |
| 3 | Hard, easily breaks |
| 4 | Hard, easily breaks |
| 5 | Hard, easily breaks |
| 6 | Hard but can be bent to make nibs |
| 7 | Hard but can be bent to make nibs |
| 8 | Hard but can be bent to make nibs |
| 9 | Hard but can be bent to make nibs |

From Table 10, it is observed that while gradual improvements were observed in terms of texture, pre-heating durations beyond 70 min (sample #6) at 80° C. were required to achieve the desired results in terms of hashish physical properties.

### Example 11: Physical Characterization of Hashish Products from Example 10

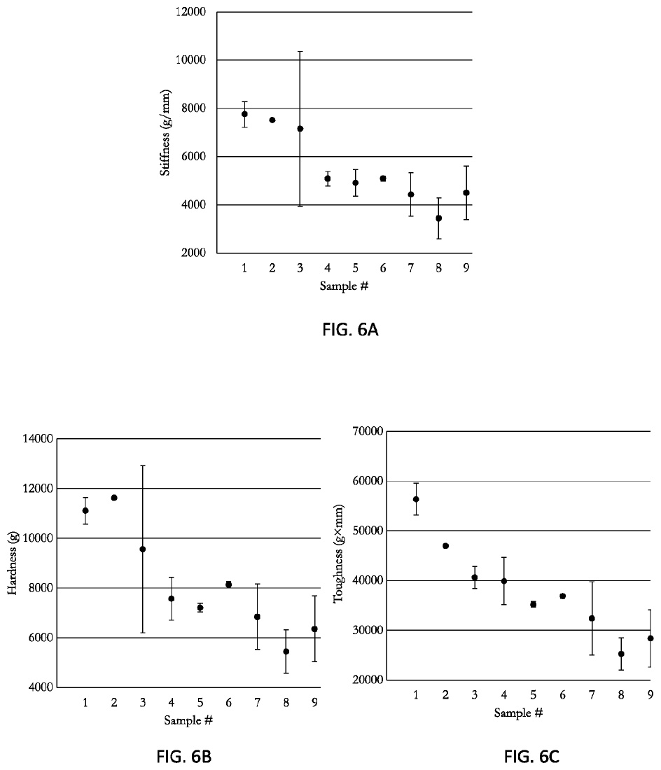

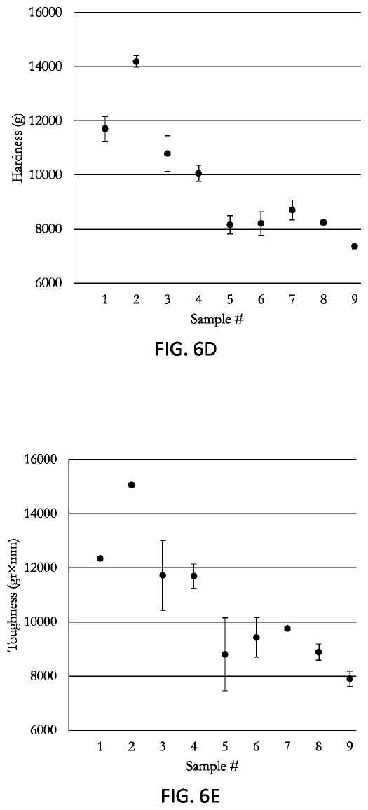

In this Example, physical properties of the hashish products manufactured in Example 10 were determined with the three-point bend test and puncture test. Results for the three-point bend test are shown in Table 11A and FIG. 6A-C.

TABLE 11A

| | Three Point Bend Test | | | |
|---|---|---|---|---|
| | | | | Sample |
| Sample | Stiffness | Hardness | Toughness | Height |
| # | (g/mm) | (g) | (g*mm) | (mm) |
| 1 | 7759 ± 535 | 11113 ± 535 | 56329 ± 3206 | 5.08 ± 0.11 |
| 2 | 7508 | 11629 | 46961 | 4.98 |
| 3 | 7156 ± 3212 | 9562 ± 3358 | 40603 ± 2273 | 5.07 ± 0.28 |
| 4 | 5087 ± 302 | 7568 ± 857 | 39885 ± 4728 | 5.09 ± 0.05 |
| 5 | 4912 ± 545 | 7215 ± 173 | 35155 ± 558 | 5.11 ± 0.25 |
| 6 | 5090 ± 100 | 8134 ± 136 | 36901 ± 443 | 5.48 ± 0.02 |
| 7 | 4427 ± 905 | 6843 ± 1314 | 32351 ± 7372 | 5.29 ± 0.14 |
| 8 | 3441 ± 848 | 5448 ± 872 | 25259 ± 3246 | 5.39 ± 0.19 |
| 9 | 4498 ± 1108 | 6357 ± 1319 | 28365 ± 5741 | 5.31 ± 0.09 |

Results for the puncture test are shown in Table 11B and FIG. 6 D-E.

TABLE 11B

| | Puncture Test | | |
|---|---|---|---|
| | Hardness | Toughness | Sample Height |
| Sample # | (g) | (g*mm) | (mm) |
| 1 | 11704 ± 462 | 12348 ± 72 | 5.42 ± 0.1 |
| 2 | 14189 ± 219 | 15063 ± 78 | 5.34 ± 0.14 |
| 3 | 10789 ± 659 | 11724 ± 1298 | 5.55 ± 0.22 |
| 4 | 10059 ± 297 | 11692 ± 453 | 5.29 ± 0.06 |
| 5 | 8160 ± 334 | 8802 ± 1339 | 5.45 ± 0.13 |
| 6 | 8203 ± 444 | 9430 ± 726 | 5.15 ± 0.17 |
| 7 | 8700 ± 370 | 9757 ± 72 | 5.34 ± 0.18 |
| 8 | 8245 ± 67 | 8894 ± 296 | 5.39 ± 0.04 |
| 9 | 7365 ± 99 | 7906 ± 276 | 5.25 ± 0.11 |

From Tables 11A-B and FIGS. 6A-E, it can be seen that even by heating the isolated cannabis trichomes at 80° C. for up to 120 min, the resulting hashish products have significantly higher stiffness, hardness and toughness values as compared to hashish products made with pre-treated isolated cannabis trichomes (e.g., samples #6-7) from example 3 or from pre-treated isolated cannabis trichomes from example 6 (samples #6-7). These results indicate that pre-treatment parameters may be controlled through monitoring of the decarboxylation level in order to obtain hashish products having desired physical properties.

### Example 12: Optimization of Pressing Parameters

In this example, a batch (Kief ID BBI-087) of isolated cannabis trichomes (NLxBB cannabis strain) was placed in an oven at 120° C. for a time duration of 25 minutes. Water was incorporated into the pre-treated isolated cannabis trichomes in the form of liquid and mixed. The resulting mixture was then pressed with a single or two-step pressing steps to obtain hashish products.

The total water content after the water incorporation step, the mould size (inches by inches), and the pressing parameters [namely: the number of press steps, the pressing load (lbs), the pressing temperature (° C.) and the pressing duration (minute or seconds)] are summarized in Table 12.

TABLE 12

| | Total water | | |
|---|---|---|---|
| Sample | content | Mould size | |
| # | (wt. %) | (in × in) | Pressing parameters |
| 1 | 9.7 | 2.5 × 2.5 | 1^{st} press: 7800 lbs/25° C./5 min, |
| | | | 2^{nd} press: 7800 lbs/70° C./20 s |
| 2 | 11.6 | 2.5 × 2.5 | 1^{st} press: 7800 lbs/25° C./5 min, |
| | | | 2^{nd} press: 7800 lbs/70° C./20 s |
| 3 | 13.5 | 2.5 × 2.5 | 1^{st} press: 7800 lbs/25° C./5 min, |
| | | | 2^{nd} press: 7800 lbs/70° C./20 s |
| 4 | 10 | 2.5 × 2.5 | 1^{st} press: 7800 lbs/25° C./5 min |
| 5 | 10 | 2.5 × 2.5 | 1^{st} press: 4200 lbs/25° C./5 min |
| 6 | 10 | 3 × 5 | 1^{st} press: 9200 lbs/25° C./5 min |

### Example 13: Visual Characterization of Hashish Products from Example 12

In this example, hashish products made in Example 12 were characterized in terms of visual and textural properties. The results are summarized in Table 13.

TABLE 13

| Sample | |
|---|---|
| # | Hashish product description |
| 1 | Acceptable malleability |
| 2 | Hashish brick was too thin for analysis (80% wasted) |
| 3 | 100% wasted product |
| 4 | Acceptable malleability |
| 5 | Acceptable malleability |
| 6 | Acceptable malleability |

Table 13 shows that while two step pressing is possible in certain circumstances, implementing a single step when pressing pre-treated isolated cannabis trichomes resulted in better malleability, and this result was achievable at various pressing loads. Further, it appears that ensuring a total water content of about 10 wt. % also affords better results in terms of malleability and also minimizes material loss following the pressing steps.

### Example 14: Physical Characterization of Hashish Products from Example 12

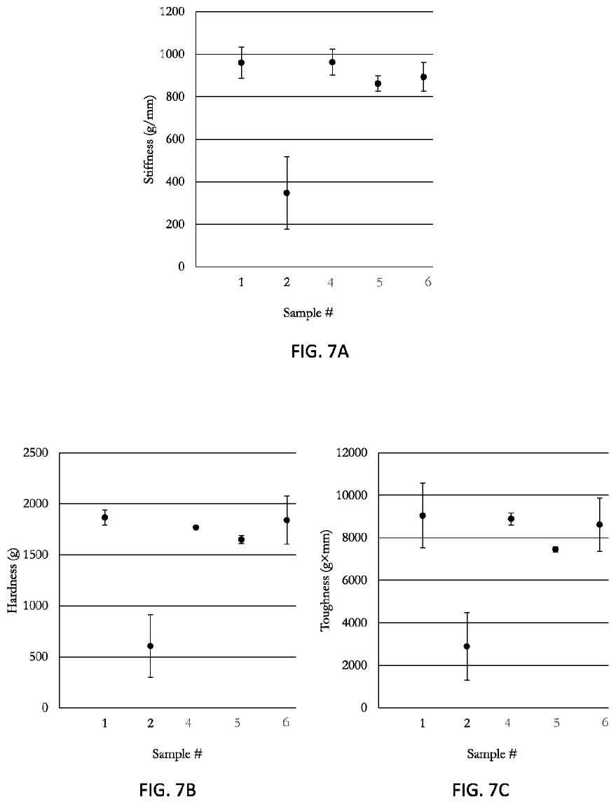

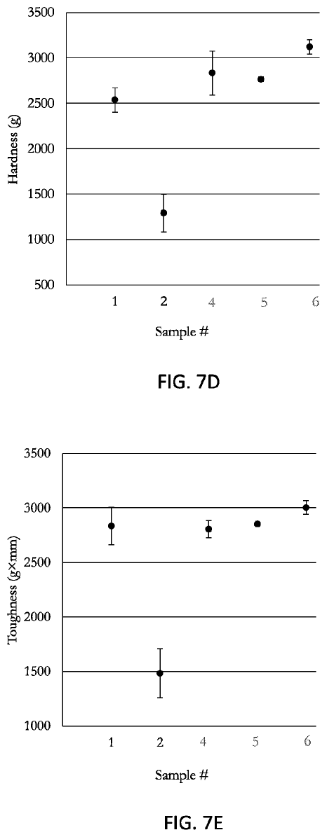

In this Example, physical properties of the hashish products manufactured in Example 12 were determined with the three-point bend test and puncture test. Results for the three-point bend test are shown in Table 14A and FIGS. 7A-C.

TABLE 14A

| | Three-Point Bend Test | | | |
|---|---|---|---|---|
| Sample | Stiffness | Hardness | Toughness | Sample Height |
| # | (g/mm) | (g) | (g*mm) | (mm) |
| 1 | 960 ± 73 | 1866 ± 73 | 9048 ± 1528 | 5.74 ± 0.09 |
| 2 | 347 ± 171 | 606 ± 307 | 2897 ± 1596 | 5.09 ± 0.02 |
| 4 | 962 ± 61 | 1768 ± 9 | 8891 ± 283 | 6.13 ± 0.01 |
| 5 | 862 ± 37 | 1650 ± 38 | 7458 ± 128 | 6.06 ± 0.13 |
| 6 | 893 ± 68 | 1840 ± 235 | 8618 ± 1253 | 6.68 ± 0.27 |

Results for the puncture test are shown in Table 14B and FIGS. 7 D-E.

TABLE 14B

| | Puncture Test | | |
|---|---|---|---|
| Sample | Hardness | Toughness | Sample Height |
| # | (g) | (g*mm) | (mm) |
| 1 | 2537 ± 133 | 2834 ± 172 | 5.46 ± 0.02 |
| 2 | 1292 ± 206 | 1484 ± 225 | 5.07 ± 0.13 |
| 4 | 2835 ± 242 | 2806 ± 78 | 5.86 ± 0.26 |
| 5 | 2765 ± 24 | 2852 ± 6 | 5.98 ± 0.17 |
| 6 | 3122 ± 79 | 3005 ± 62 | 6.65 ± 0.16 |

Tables 14A-B and FIGS. 7A-E show that pressing pre-treated isolated cannabis trichomes with two or a single pressing step provided hashish products with desired stiffness, hardness and toughness, as measured in the three-point bend test. The resulting hashish products have one or more of the following: a higher limit of stiffness of about 3200 g/mm, a higher limit of hardness of about 2500 g, and a higher limit of toughness of about 8000 g*mm, as measured in the three-point bend test.

Other examples of implementations will become apparent to the reader in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

Reference throughout the specification to “some embodiments”, and so forth, means that a particular element (e.g., feature, structure, and/or characteristic) described in connection with the invention is included in at least one embodiment described herein, and may or may not be present in other embodiments. In addition, it is to be understood that the described inventive features may be combined in any suitable manner in the various embodiments.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art, such as for example+/−20%, +/−15%, +/−10%, or +/−5%. Hence, numerical quantities given herein generally include such error margin such that the terms “around”, “about” or “approximately” can be inferred if not expressly stated.

As used throughout the present disclosure, the terms “concentration” and “content” are used interchangeably and refer to the weight or mass fraction of a constituent, i.e., the weight or mass of a constituent divided by the total mass of all constituents, and is expressed in wt. %, unless stated otherwise.

Although various embodiments of the disclosure have been described and illustrated, it will be apparent to those skilled in the art considering the present description that numerous modifications and variations can be made. The scope of the invention is defined more particularly in the appended claims.

1. A process for making a hashish product, comprising
a) providing pre-treated isolated cannabis trichomes, the pre-treated isolated cannabis trichomes comprising a cannabis oil layer on at least a portion of a surface thereof; and
b) pressing the pre-treated isolated cannabis trichomes under conditions to form the hashish product, the hashish product being a cohesive mass of the pre-treated isolated cannabis trichomes, and the hashish product comprising a not negligible content in acid form of one or more cannabinoid(s).

2. The process according to claim 1, wherein the step a) comprises pre-heating isolated cannabis trichomes under conditions to obtain partial decarboxylation of the one or more cannabinoid(s).

3. The process according to claim 1, wherein the step a) comprises (i) pre-heating cannabis material under conditions to obtain partial decarboxylation of the one or more cannabinoid(s) and (ii) isolating cannabis trichomes therefrom.

4. The process according to claim 2, wherein the pre-heating is performed at a temperature of from about 70° ° C. to about 130° C., preferably from about 80° C. to about 120° C., more preferably about 120° C.

5. The process according to claim 2, wherein the pre-heating is performed for a duration of from about 10 minutes to about 60 minutes, preferably from about 20 minutes to about 35 minutes.

6. The process according to claim 2, wherein the pre-heating is performed to obtain a decarboxylation level of the one or more cannabinoid(s) of from about 30% to about 90%, preferably from about 40% to about 80%, more preferably from about 50% to about 70%, even more preferably about 60%.

7. The process according to claim 2, wherein the pre-heating is performed to obtain a ratio of a decarboxylated to acid form content of the one or more cannabinoid(s), by weight, of from about 100:1 to about 1:100, preferably from about 1:4 to about 50:1, more preferably from about 1:3 to about 5:1.

8. The process according to claim 1, wherein the not negligible content in the acid form of the one or more cannabinoid(s) is of no less than 1 wt. %, preferably of no less than 3 wt. %, even more preferably of no less than 5 wt. %.

9. The process according to claim 8, wherein the not negligible content in the acid form of the one or more cannabinoid(s) is of up to about 30 wt. %, preferably up to about 25 wt. %, more preferably up to about 20 wt. %.

10. The process according to claim 1, wherein the acid form of the one or more cannabinoid(s) comprises tetrahydrocannabinolic acid (THC-A), cannabidiolic acid (CBD-A), or THC-A and CBD-A.

11. The process according to claim 1, further comprising incorporating water to the pre-treated isolated cannabis trichomes prior to step b) to have a water content of about 20 wt. % or less, preferably between about 5 wt. % and about 15 wt. %, more preferably from about 10 wt. % to about 15 wt. %.

12.-14. (canceled)

15. The process according to claim 11, wherein the pressure is from about 100 psi to about 3200 psi, preferably from about 300 psi to about 1500 psi, more preferably from about 500 psi to about 1250 psi.

16. The process according to claim 1, wherein the pressing is performed for a time duration of about 1 minute or more, preferably from about 1 minute to about 10 minutes, more preferably from about 3 minutes to about 7 minutes, even more preferably from about 4 minutes to about 6 minutes.

17.-18. (canceled)

19. The process according to claim 1, wherein the pre-treated isolated cannabis trichomes are pre-treated kief.

20. A hashish product comprising a cohesive mass of isolated cannabis trichomes and a not negligible content in acid form of one or more cannabinoid(s) and made by the process according to claim 1.

21. A hashish product comprising a cohesive mass of isolated cannabis trichomes, a not negligible content in acid form of one or more cannabinoid(s) and having one or more of the following properties as determined in a three-point bending test:
a) a higher limit of stiffness of about 3200 g/mm,
b) a higher limit of hardness of about 2500 g, and
c) a higher limit of toughness of about 8000 g*mm.

22. The hashish product according to claim 21, wherein the acid form of the one or more cannabinoid(s) comprises tetrahydrocannabinolic acid (THC-A), cannabidiolic acid (CBD-A), or both THC-A and CBD-A.

23. The hashish product according to claim 21, wherein the not negligible content in the acid form of the one or more cannabinoid(s) is of no less than 1 wt. %, preferably of no less than 3 wt. %, even more preferably of no less than 5 wt. %.

24. The hashish product according to claim 23, wherein the not negligible content in the acid form of the one or more cannabinoid(s) is of up to about 30 wt. %, preferably up to about 25 wt. %, more preferably up to about 20 wt. %.

25.-26. (canceled)

27. The hashish product according to claim 21, wherein the isolated cannabis trichomes are kief.
